(19) United States
(12) Patent Application Publication
(10) Pub. No.: US 2019/0145957 A1
(43) Pub. Date: May 16, 2019

(54) METHODS FOR EVALUATING THE PROTECTION EFFICACY OF A SUNSCREEN AGENT

(71) Applicant: NESTLE SKIN HEALTH S.A., Lausanne (CH)

(72) Inventors: Annalisa STILLA, Egerkingen (CH); Nicole WALTER, Egerkingen (CH); Nicola HEWITT, Egerkingen (CH); Gernot KUNZE, Egerkingen (CH)

(73) Assignee: NESTLE SKIN HEALTH S.A., Lausanne (CH)

(21) Appl. No.: 16/097,061

(22) PCT Filed: Apr. 28, 2017
(86) PCT No.: PCT/EP2017/060178

§ 371 Date: Oct. 26, 2018

Related U.S. Application Data
(60) Provisional application No. 62/329,997, filed on Apr. 29, 2016.

(51) Int. Cl. G01N 33/50; G01N 21/33

(52) U.S. Cl. CPC G01N 33/5008; A61Q 17/04; G01N 21/33

(57) ABSTRACT

The present disclosure is directed a method for evaluating a sunscreen. The method comprises measuring a protective effect of a sunscreen and at least one cellular alteration caused by irradiation. The measured effects are evaluated against a control for the at least one cellular alteration caused by irradiation.

### FIELD

The present disclosure relates to methods for evaluating a sunscreen and related methods for measuring the protective effects of a sunscreen agent against one or more cellular alterations caused by irradiations, in particular UV irradiations.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

Ultraviolet (UV) radiation exposure from the sun and artificial UV sources has been widely acknowledged as the major cause for skin cancer and premature skin aging¹. Penetration of UV radiation into skin is wavelength-dependent and leads to different biological effects, such as erythema, DNA damage, immune suppression and formation of free radicals, leading to oxidative damage of DNA and other biomolecules. UVB mainly affects the stratum corneum and the top layers of the epidermis² where it is absorbed by epidermal components such as proteins and DNA, with only 10 to 15% of the radiation reaching the dermis³. UVA radiation penetrates deeply into the skin and reaches the lower epidermis and dermal fibroblasts where it can induce other long-term biological effects mainly due to oxidative damage to skin cell components¹. Therefore, adequate photo-protection such as seeking shade, wearing protective clothing and using sunscreens is the key to reducing the harmful effects of UV radiation.

Sunscreens have become a quasi-exclusive mode of protection and all consist of a combination of UV absorbers and a carrier system (vehicle) into which they are incorporated. The main goal in the development of a sunscreen product is to achieve highest efficacy protection by selecting an optimal combination of UV absorbers. Currently, in vitro sunscreen tests are used to examine the UVA-PF and the photo-stability of the chemicals/absorbers⁴⁻⁶. These involve specialized spectrophotometric measurements of the absorbance of UV radiation through a sunscreen applied on a suitable substrate (e.g. polymethylmethacrylate (PMMA) or quarz plates) and allow an evaluation of the protection capability/efficacy both at short (290-320 nm, UVB) and long (320-400 nm, UVA) UV wavelengths. Additional information on the damaging effects of UV radiation in biological substrates, ideally collected under similar standardized in vitro sunscreen testing (using PMMA plates), would potentially complement the spectrophotometric measurements with biologically relevant information to increase the meaning of sunscreen product characterization during the development process and might be of use even for marketed products. Thus, there is a need for biological markers suitable for the characterization of UV-induced damage at the cellular level.

### SUMMARY

According to certain aspects of the disclosure potential biological markers suitable for the characterization of UV-induced damage at the cellular level were selected. Three key target/pathways of molecular effects of UV radiation, as well as their direct cytotoxic effects, namely, cyclobutane pyrimidine dimer (CPD) formation, p38 phosphorylation, p53 activation and membrane leakage were of focus.

Solar ultraviolet (UV) radiation is the main cause of changes leading to skin damage, such as sunburn, erythema, skin photo-aging. Applicant has developed an in vitro model that combines the use of skin cells such as keratinocytes, fibroblasts, melanocytes or mixture thereof, for example of cell cultures, typically of keratinocyte cultures; skin sample; skin model; or reconstituted skin, and sunscreen-coated PMMA or quarz plates to measure the protective effects of a panel of 15 sunscreens against a number of selected cellular alterations caused by UVB and UVA irradiation. Endpoints include, but are not limited to, cell vitality (membrane leakage in early apoptosis and Trypan blue exclusion), as well as the measurement of cyclobutane pyrimidine dimers (CPDs) formation, p38 phosphorylation and p53 activation. The optimal time at which each measurement was critical varied between 30 min and 6 h. The analysis of different formulations with combinations of UV absorbers and different Sun Protection Factor (SPFs) showed that a good degree of protection is provided by formulations containing UVB filters and, in general, the degree of protection correlates well with the spectral absorption curve of the tested formulations. Although very high and high protection sunscreens always afforded nearly 100% protection against the endpoints measured, the degree of protection was not directly correlated with the SPF. Sunscreen formulations containing only UVA filters did not provide complete protection, indicating that these specific endpoints are mainly affected by UVB, but partly also by UVA. The present invention provides methods which can be advantageously used in the initial screening of active ingredients in Sunscreens.

Herein described in particular is an in vitro, ex vivo or in vivo method of evaluating a sunscreen comprising (a) measuring a protective effects of a sunscreen and at least one cellular alteration caused by UV irradiation, typically UVB and/or UVA irradiation, and (b) evaluating the sunscreen by comparing the protective effects thereof against a control, wherein the comparison is based on the at least one cellular alteration. When performed in vitro, the method is typically performed on skin related cells such as keratinocytes culture(s), preferably using sunscreen-coated PMMA or quarz plate(s). The measure of at least one cellular alteration preferably involves the measure of an endpoint which is typically selected from at least one of cyclobutane pyrimidine dimer (CPD), p38 protein (preferably phosphorylated p38), cell viability and p53 protein. The measure is typically performed via an enzyme-linked immunosorbent assay (ELISA) and/or fluorescence activated cell sorting (FACS) method.

Also herein described is an in vitro, ex vivo or in vivo method of evaluating a sunscreen, typically the protective effects of a sunscreen against cellular changes caused by irradiation, typically ultraviolet (UV), visible (VIS) and/or infra-red (IR), typically IR-A, comprising:

(a) measuring protective effect(s) of a sunscreen and at least one cellular alteration caused by irradiation over the UV up to IR wavelengths, typically over the UV, visible and IR wavelengths, and
(b) evaluating the sunscreen by comparing the protective effect(s) thereof against a control, wherein the comparison is based on the at least one cellular alteration.

The method typically comprises a step of exposing (“irradiation step”) cells to irradiation. The irradiation step is typically performed with an irradiation source emitting radiations selected from ultraviolet (UV), visible (VIS) and/or infra-red (IR), typically IR-A, typically radiations having wavelengths ranging from 250 to 850 nm or expressed in J/cm².

In a particular aspect, the irradiation is a light irradiation (also herein identified as “visible” irradiation or “VIS” irradiation).

In another particular aspect, the irradiation is an infra-red (IR) irradiation. The irradiation is preferably a UV irradiation, typically a UVB and/or a UVA irradiation, preferably a UVB irradiation.

Protective effect(s) of a sunscreen and cellular alteration(s) are to be observed on cells, typically on a cellular tissue, cell culture or cell model. In the herein described methods cells comprises keratinocytes, fibroblasts, melanocytes or any mixture thereof, and are preferably keratinocytes. Cells can be typically a culture of keratinocytes, such as HaCaT cells or NHEK cells, or a cellular model comprising keratinocytes. In a particular method herein described, a sunscreen-coated substrate is advantageously placed between the surface of cells and the light irradiation source during light irradiation. The substrate is preferably a polymethylmethacrylate (PMMA) or quarz plate. The substrate is preferably coated with 0.50 mg up to 1.5 mg sunscreen per cm², typically from 0.50 mg up to 1.3 mg sunscreen per cm², for example 0.50, 0.55, 0.60, 0.65, 0.70, 0.75, 0.80, 0.85, 0.90, 0.95, 1.00, 1.05, 1.10, 1.15, 1.20, 1.25 or 1.30 mg sunscreen per cm², preferably 0.75 mg sunscreen per cm².

In the methods herein described, step (a) preferably comprises detecting, measuring or monitoring at least two, preferably three, even more preferably four, cellular alterations selected from cyclobutane pyrimidine dimer (CPD) formation, p38 protein phosphorylation, p38 mitogen-activated protein kinase (MAPK) activation, p53 protein activation and cell viability or intermediate stages of apoptosis. Step (a) may be performed several times with respect to a particular sunscreen to test.

When the cellular alteration to detect or measure is CPD formation, the detection or measure is preferably to be performed between 2 hours and 15 hours post-irradiation, the irradiation being preferably a 828 mJ/cm² irradiation or less. When the cellular alteration to detect or measure is CPD formation, the detecting or measuring step is typically performed by ELISA or FACS, preferably by ELISA.

when the cellular alteration to detect or measure is p38 protein phosphorylation or p38 mitogen-activated protein kinase (MAPK) activation, the detection or measure is preferably to be performed from immediately after irradiation up to two hours post-irradiation, even more preferably 30 minutes post-irradiation, the irradiation being preferably a 200 mJ/cm² irradiation. When the cellular alteration to detect or measure is p38 protein phosphorylation or p38 mitogen-activated protein kinase (MAPK) activation, the detecting or measuring step is typically performed by FACS.

When the cellular alteration to detect or measure is p53 protein activation, the detection or measure is preferably to be performed 6 hours post-irradiation, the irradiation being preferably a 100 mJ/cm² irradiation.

When the cellular alteration to detect or measure is p53 protein activation, the detecting or measuring step is typically performed by ELISA.

When the cellular alteration to detect, measure or monitor is cell viability or intermediate stage(s) of apoptosis, the detection or measure is preferably to be performed from 1 hour up to 30 hours post-irradiation, even more preferably 24 hours post-irradiation, once or several times, the irradiation being preferably an irradiation above 75 mJ/cm², even more preferably a 828 mJ/cm² irradiation.

When the cellular alteration to detect, measure or monitor is cell viability, the detecting, measuring or monitoring step is performed by tryptan blue exclusion.

When the cellular alteration to detect, measure or monitor is an intermediate stage of apoptosis, the detecting, measuring or monitoring step is performed by measure of the externalization of phosphatidyl serine.

Anyone of the herein described methods can be advantageously performed in parallel on several identical or different keratinocytes cultures or models comprising keratinocytes, with identical or different sunscreens, typically with sunscreens comprising different UVA and/or UVB filters or absorbers or different concentrations of such UVA and/or UVB filters or absorbers. The method can be performed in parallel under different irradiation conditions. The method can be performed at one or different time points.

### BRIEF DESCRIPTION OF THE DRAWINGS

These and other features of this invention will now be described with reference to the drawings of certain embodiments which are intended to illustrate and not to limit the disclosure.

FIG. 1 depicts a graph of the spectral irradiance of UVB and UVA irradiation (black line), irradiance measured in Albuquerque on Jul. 3, 2002 (dark grey line); and irradiance measured in Melbourne on Jan. 17, 1990 (light grey line).

FIG. 2 depicts a graph of the effects of different doses of UV irradiation on the viability and percentage of apoptotic cells. Viability (▪) and % apoptotic cells (∘) in HaCaT cultures 2 h (●) and 24 h (□) post-irradiation at different UV doses was expressed as a percentage of control cells (without UV irradiation). Values are mean±SD of at least 3 independent experiments performed in triplicate.

FIG. 3 depicts a graph of the effects of UV irradiation on CPD formation. Dose effects (A): HaCaT cells were exposed to UV (from 20 mJ/cm² to 828 mJ/cm2) and then incubated for 2 h. CPDs were analyzed by FACS (∘) or ELISA (●).

Time-dependent effects (B): HaCaT cells were exposed to 50 mJ/cm² (∘) or 828 mJ/cm² (●) UV irradiation and then incubated for up to 15 h. Values are expressed as a fold of control levels, mean±SD of at least two independent experiments performed in triplicate, statistical differences from control values are denoted with an asterisk (*).

FIG. 4 depicts a graph of the effects of UV irradiation on p38 phosphorylation. Time-dependent effects (A): HaCaT cells were exposed to UV (200 mJ/cm²) and further incubated for up to 2 h.

Dose effects (B): HaCaT cells were exposed to UV (from to 20 to 828 mJ/cm2) and further incubated for 30 min. The phosphorylated p38 protein was analysed by FACS. Values are expressed as a fold of control levels, mean±SD of at least two independent experiments performed in triplicate, statistical differences from control values are denoted with an asterisk (*).

FIG. 5 depicts a graph of the effects of UV irradiation on p53 activation. Time-dependent effects (A): NHEK cells were exposed to 50 mJ/cm² UV irradiation and then incubated in serum-free media for up to 24 h.

Dose effects (B): NHEK cells were exposed to UV (from 50 to 100 mJ/cm²) and then incubated for 6 h. The p53 protein was analysed by ELISA. Values are expressed as a fold of control levels, mean±SD of three independent experiments performed in triplicate, statistically higher differences from control values are denoted with an asterisk (*).

FIG. 6 depicts a graph of the protective effects of different sunscreens from cytotoxicity induced by UV.

HaCaT cells were exposed to 828 mJ/cm² UV with and without PMMA plates coated with sunscreen and then further incubated for 24 h. Values are expressed as percentage control viability, measured by Trypan blue exclusion; mean±SD of at least 3 independent experiments performed in triplicate. An asterisk (*) indicates a statistically significant difference from control cell viability (P<0.05).

FIG. 7 depicts a graph of the protective effects of different sunscreens from CPD formation induced by UV.

HaCaT cells were exposed to 828 mJ/cm² UV with and without PMMA plates coated with sunscreen and then further incubated for 2 h. Values are expressed as percentage inhibition of CPD formation; mean±SD of 5 independent experiments performed in triplicate. An asterisk (*) indicates a statistically significant difference from SS1-VH (P<0.05).

FIG. 8 depicts a graph of the UV-absorption spectra generated by using the Colipa in vitro UVA-protection method.

UV-Absorption Spectra (after irradiation) generated by using the Colipa in vitro UVA-protection method (2011) with the test sunscreens, (A) SS1-VH (black line) and SS11-M (grey line), (B) SS9-H (black line) and SS10-M (grey line) and (C) SS8-H (dark grey line), SS9-H (light grey line), SS13-L (solid black line) and Colipa P3 (dashed black line).

FIG. 9 depicts the experimental set-up for the measurement studies.

Photos showing how PPMA plates are placed on top of the keratinocyte culture plates (A) and then placed in the irradiation source equipment (B). This model uses PMMA plates as a support for the sunscreens that are placed between the cells and the UV irradiation source. After UV exposure in the presence and absence of sunscreens, UV-induced endpoints are analyzed in the keratinocytes. The amount of sunscreen applied to the PMMA plates is linked to the amount used to measure the SPF in vivo and the UVA-PF using the validated in vitro assay. Furthermore, the UV doses used were in a range relevant to human solar light exposure (828 mJ/cm² in the experimental conditions, equivalent to 6MED (minimal erythema dose). Keratinocytes (contrastive to human skin, without a protective stratum corneum) were employed because they are the first layer of living cells exposed both to UVB and UVA radiation and have an inherent antioxidant defense mechanism against oxidative stress 16.

FIG. 10 depicts a table (also herein identified as table 1) of the category/classification, composition, sun protection values for the sunscreens tested.

### DETAILED DESCRIPTION

Further aspects, features and advantages of this invention will become apparent from the detailed description which follows. All patents and technical references referenced herein are incorporated by reference in their entireties.

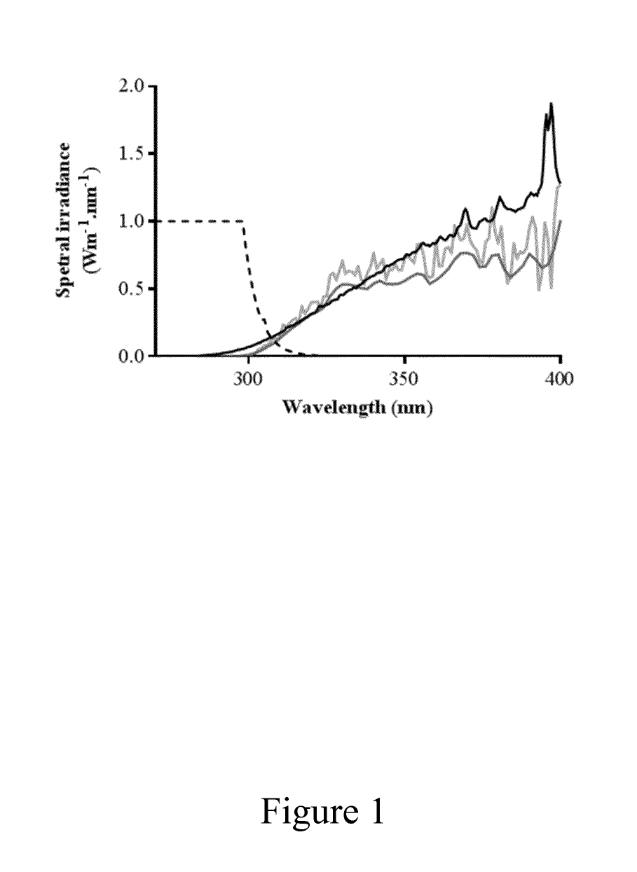

According to certain aspects of the disclosure potential biological markers suitable for the characterization of UV-induced damage at the cellular level were selected. Applicant focused on three key targets/pathways of molecular effects of UV radiation, as well as its direct cytotoxic effects, namely, cyclobutane pyrimidine dimer (CPD) formation, p38 phosphorylation and p53 activation, membrane leakage being advantageously assessed in addition to anyone of the three previously mentioned cytotoxic effects. CPD formation and other DNA damage results from DNA directly absorbing UVB⁸⁻⁹. Although UV-induced CPDs can be repaired, they are considered responsible for the vast majority of carcinogenic mutations. UV irradiation activates p38, which is involved in mediating both cellular survival and death in UV-irradiated epidermal keratinocytes and HaCaT cells⁸⁻⁹. The gene suppressor factor, p53, participates in DNA repair through the control of cell cycle check-points. This functional pathway is of importance because mutations in p53 are often found in human and animal skin cancer cells¹⁰. Dermal alterations related to penetration of UVA radiation through the fibroblast-containing dermis are not herein described, but the same principle can be applied to other 2D and 3D epidermal and full-thickness models. Applicant conducted a series of experiments to optimize and explore the application of a novel experimental approach that combines some features of the well-accepted in vitro COLIPA method⁴ (an EU precursor guideline of actual IS024443:2012⁵) with cellular endpoint measurements in keratinocytes. The light source emitted both UVB and UVA irradiation and its irradiance spectrum was similar to that measured in Albuquerque (38° N) at noon and in Melbourne (38° S) at solar noon¹¹ (FIG. 1) and therefore represents actual UV exposure to humans. To evaluate the experimental setting, a panel of 15 sunscreen products with SPFs ranging from 5 to 50+(based on EU classification) were tested using this model. The filter composition and the main features of the tested sunscreens are listed in Supplemental Table 1 and Table 1 (Table appearing on FIG. 10), respectively.

SUPPLEMENTAL TABLE 1
sunscreen compositions of UV absorbers (which are to be considered as
examples only) and corresponding sun protection values.

| | Product classification* according to EU recommendation¹³ | | | | | | | |
|---|---|---|---|---|---|---|---|---|
| | VERY HIGH | VERY HIGH | VERY HIGH | HIGH | HIGH | HIGH | HIGH | HIGH |
| | Type of Formulation** | | | | | | | |
| | W/O | | | | | | | |
| | [symbol] | W/O | W/O | W/O | W/O | W/O | W/O | W/O |
| | Qualitative absorber composition | | | | | | | |
| | SS1-VH | SS2-VH | SS3-VH | SS4-H | SS5-H | SS6-H | SS7-H | SS8-H |
| ZnO₂ | | | | | | | | |
| TiO₂ | | ✓ | | ✓ | | | | ✓ |
| Avobenzone | ✓ | ✓ | ✓ | ✓ | | | | ✓ |
| Tinosorb S | ✓ | ✓ | ✓ | | ✓ | | ✓ | ✓ |
| Tinosorb M | ✓ | | ✓ | | ✓ | | | |
| Octinoxate | ✓ | ✓ | | | ✓ | | | ✓ |
| Octocrylene | | ✓ | | ✓ | | | | ✓ |
| Uvinul A Plus | | ✓ | | | | ✓ | | |
| Uvasorb HEB | | ✓ | | | | ✓ | | ✓ |
| Sulisobenzone | | | | | | | ✓ | |
| Enzacamene | | | | | | | | |
| Mexoryl SX/Ecamsule | | | | ✓ | | | | |
| Mexoryl XL/ | | | | ✓ | | | | |
| Drometrizole Trisiloxane | | | | | | | | |
| Uvinul T 150 | ✓ | | | | ✓ | ✓ | | |
| Amiloxate | ✓ | | | | | | | |
| Ensulizole | | ✓ | | | | | | |
| Iscotrizinol | | | ✓ | | | | | |
| Octisalate | | | ✓ | | | | | |

| | Product classification* according to EU recommendation¹³ | | | | | | |
|---|---|---|---|---|---|---|---|
| | HIGH | MEDIUM | MEDIUM | MEDIUM | LOW | LOW | // |
| | Type of Formulation** | | | | | | |
| | W/O | W/O | W/O | W/O | W/O | W/O | W/O |
| | Qualitative absorber composition | | | | | | |
| | | | | | Colipa | | SS14- |
| | SS9-H | SS10-M | SS11-M | SS12-M | [symbol] | SS13-L | [symbol] |
| ZnO₂ | | | | ✓ | | | |
| TiO₂ | | | ✓ | | | | |
| Avobenzone | ✓ | ✓ | ✓ | | ✓ | | |
| Tinosorb S | ✓ | ✓ | | | | | |
| Tinosorb M | | | | | | | |
| Octinoxate | ✓ | | ✓ | ✓ | ✓ | | |
| Octocrylene | | | | | | | |
| Uvinul A Plus | | | | | | | ✓ |
| Uvasorb HEB | | | | | | ✓ | |
| Sulisobenzone | | | | | | | |
| Enzacamene | | | ✓ | ✓ | | | |
| Mexoryl SX/Ecamsule | | | | | | | |
| Mexoryl XL/ | | | | | | | |
| Drometrizole Trisiloxane | | | | | | | |
| Uvinul T 150 | ✓ | ✓ | | | | ✓ | |
| Amiloxate | | | | | | | |
| Ensulizole | | | | | ✓ | | |
| Iscotrizinol | | | | | | | |
| Octisalate | | ✓ | | | | | |

*Product classification according to published guideline of the European Commission in the official journal of the European Union “Recommendation on the efficacy of sunscreen products and the claims relating thereto”¹³
**All formulations are typical oil/water formulations; no sprays, no gels have been included in the evaluations; however, the latter formulations have been evaluated with the same approach in subsequent studies (not published). [symbol]
indicates data missing or illegible when filed

Results

Optimization of the Measurement of Cellular Endpoints

Viability and Apoptosis in UV-Irradiated HaCaT Cells

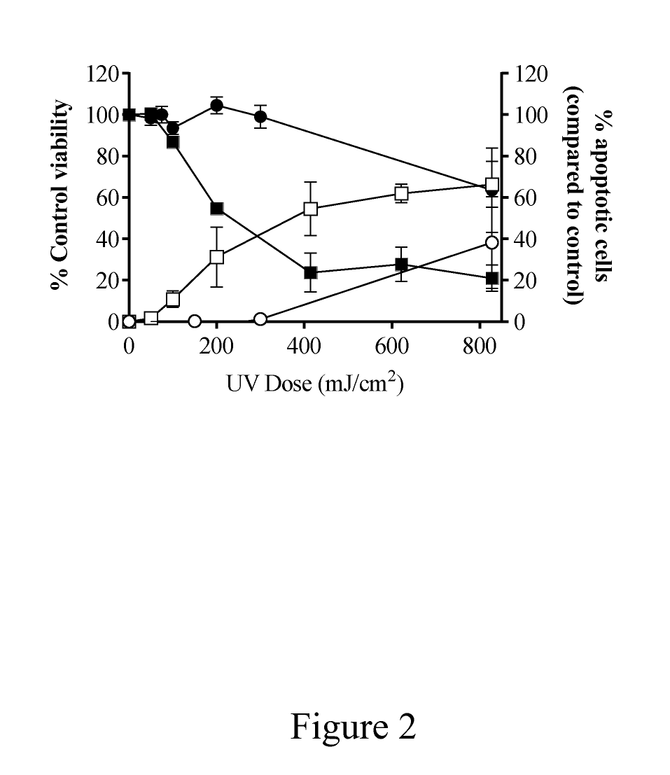

As shown herein, FIG. 2 shows the effects of UV irradiation (including UVB and UVA) and subsequent post-irradiation times on the viability of HaCaT cultures and the % of apoptotic cells. Viability, measured using Trypan blue exclusion, correlated well with the percentage of cells undergoing apoptosis, such that an increase in the % apoptotic cells resulted in a decrease in membrane integrity. All cells were viable and there were no signs of apoptosis after receiving a dose of 50 mJ/cm² (equivalent to 0.4 MED (minimal erythemal dose)) but higher doses resulted in marked apoptosis and a loss of culture viability. At high doses (400 to 800 mJ/cm²), it is likely that only late apoptotic/necrotic cells were present 24 h post-irradiation. Maximal cell death and apoptosis 24 h post-irradiation occurred between 400 and 828 mJ/cm². Since some of the experiments described below were performed 2 h post-irradiation, we were also interested to know the toxic effects of UV at this time point. As shown in FIG. 2, there was minimal cell death or apoptosis observed at doses up to 300 mJ/cm² 2 h post-irradiation. At 828 mJ/cm² (equivalent to 6 MED), only 64% of the cells were still viable according to Trypan blue exclusion and 38% of the cells showed signs of apoptosis. These results were used as a dose-finding guide, in order to evaluate other cellular endpoints at doses that caused the least cytotoxicity or apoptosis. Optimal doses and time points are described for each endpoint below.

CPD Formation in UV Irradiated HaCaT Cells

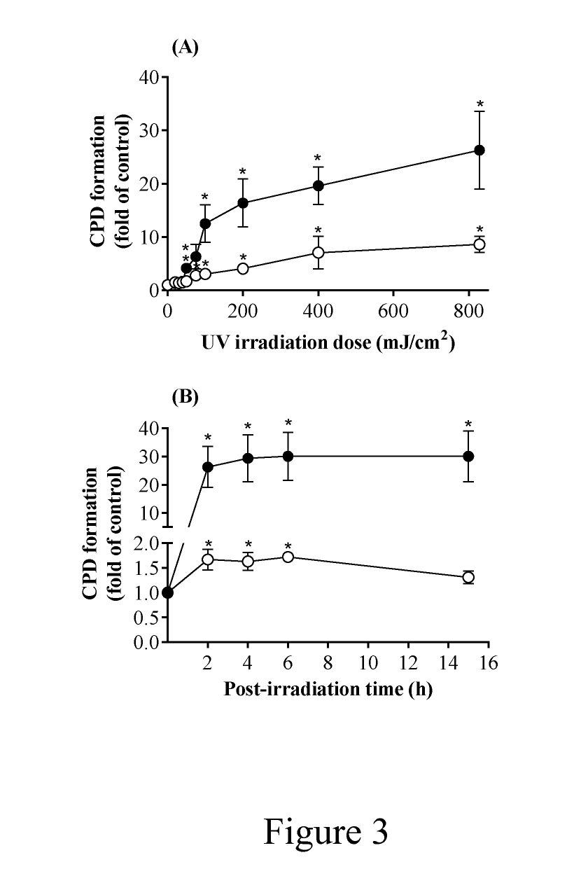

The UV-induced CPDs in HaCaT cells were measured both by enzyme-linked immunosorbent assay (ELISA), using purified DNA from irradiated cells, and by fluorescence activated cell sorting (FACS). The FACS and ELISA methods produced similar profiles of CPD formation; however, the FACS method was found to be less sensitive (FIG. 3A). For this reason, only ELISA results are reported for time-dependent studies and the effects of sunscreens on this endpoint. This assay is specific for CPDs since specific antibodies against CPDs were used and these would not detect simple DNA breaks (which can be detected using the comet assay depending on the pH used for alkalinisation).

The kinetics of repair of UVB-induced DNA lesions in HaCaT cells has been reported to slow with increasing doses of UVB¹². Inventors discovered that there was a dose-dependent increase in the formation of CPDs 2 h post irradiation at non-cytotoxic doses between 50 and 200 mJ/cm² (by 4- and 15-fold respectively, FIG. 3A). The kinetics of the formation of CPDs was investigated after non-toxic and toxic doses of 50 and 828 mJ/cm² UV radiation, respectively (FIG. 3B). A dose of 828 mJ/cm² caused more than a 20-fold increase of CPDs compared to control cells, an effect that was evident 2 h post-irradiation. A non-toxic dose of 50 mJ/cm² also caused an increase in CPD formation after only 2 h but the increase was significantly lower. For both UV doses, the number of CPDs did not increase further after this time but the presence of CPDs was persistent and in cells exposed to 828 mJ/cm², they were detected even after 15 h post-irradiation (FIG. 3B). The number of CPDs decreased to control levels between 6 h and 15 h after a dose of 50 mJ/cm², which was likely due to DNA repair since this dose was not toxic and did not cause apoptosis at any time point.

Based on these findings and in order to compromise between the number of viable, non-apoptotic, cells and maximise detectable CPD formation, subsequent experiments investigating the effects of sunscreens employed a dose of 828 mJ/cm² (equivalent to 6 MED) and a 2 h post-irradiation time point.

UVB Induced p38 Phosphorylation in HaCaT Cells

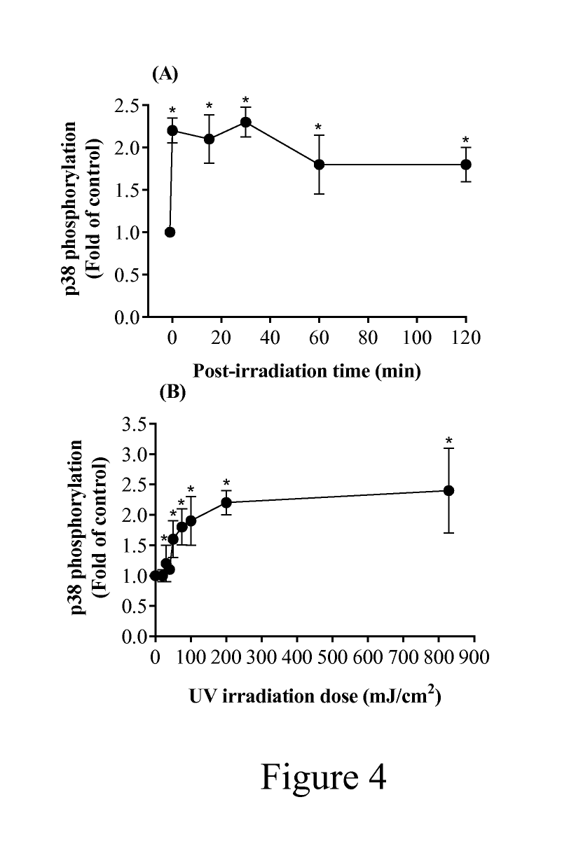

FIG. 4A shows the time course and extent of phosphorylation of p38 to its enzymatically active form in HaCaT cells exposed to 200 mJ/cm² UV over 2 h post-irradiation. Exposure of HaCaT cells to UV (200 mJ/cm²) resulted in marked phosphorylation of p38 compared to control cells (without UV irradiation), which was detected immediately after UV irradiation and was persistent over 1 h post-irradiation. After this time, p38 activation declined slowly (FIG. 4A). In order to capture the optimal conditions for this effect 30 min was selected as the optimal post-irradiation experimental time point. The maximum activation of p38 phosphorylation obtained for 200 mJ/cm² under these conditions was 2.4-fold. At lower irradiation doses (50 mJ/cm²), only a slow and non-reproducibly significant activation of p38 phosphorylation was observed. At higher irradiation doses at which saturation was reached, a higher variability in the rate of phosphorylation of p38 was observed (FIG. 4B), as well as a lower number of viable cells. For this reason, all subsequent experiments with sunscreens were carried at a dose of 200 mJ/cm² (equivalent to 1.4 MED) and analysed 30 min post-irradiation.

UVB Induced p53 Induction in NHEK Cells

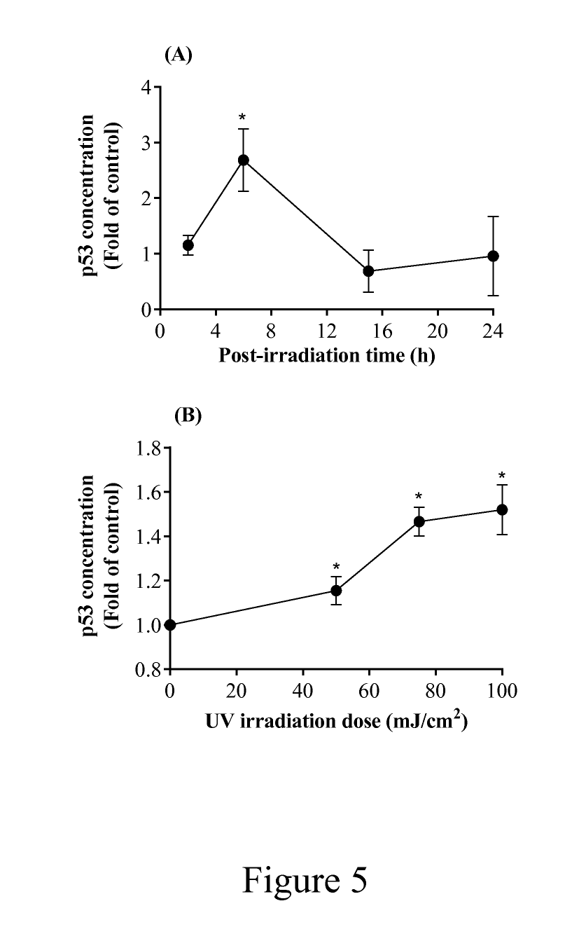

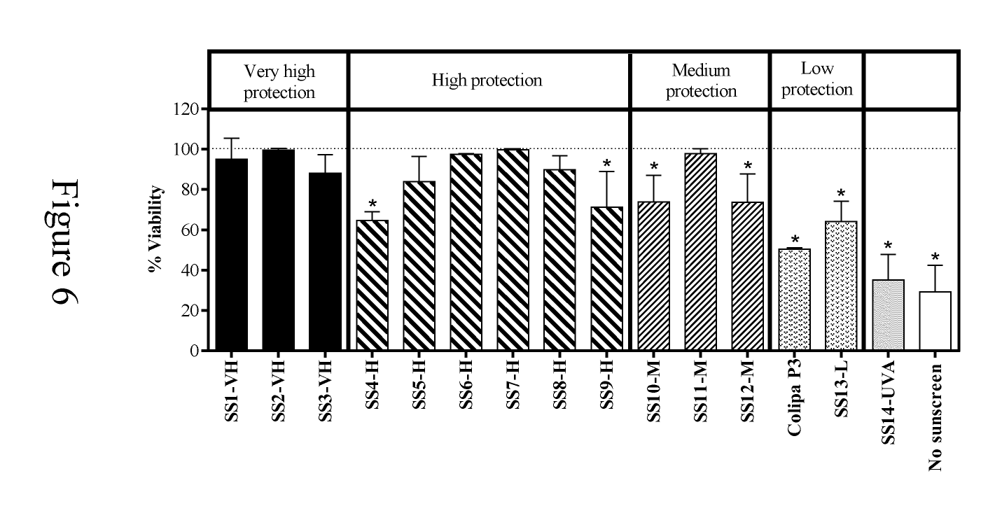

FIG. 5A shows the effect of a UV dose of 50 mJ/cm² on the activation of p53 over 24 h post irradiation. The window in which activation was measurable was short, such that a significant increase (2.7-fold) was only evident at the 6 h post irradiation time point. In order to achieve a maximal response, the effect of UV doses between 50 and 100 mJ/cm² were measured 6 h post-irradiation (FIG. 5B). There was a dose-dependent increase in the activation of p53 which reached a maximal level at 100 mJ/cm². The fold increases were smaller in these experiments, mainly due to a higher control expression of p53 in the batch of cells used for the dose response experiments (31.3±5.3 pg/ml) than in those used for the time course experiments (12.5±6.6 pg/ml). Based on these studies, the optimal conditions for evaluating the protective effects of sunscreens were set at 100 mJ/cm² (equivalent to 0.7 MED) measured 6 h post-irradiation.

Effect of Sunscreens on Cellular Endpoints

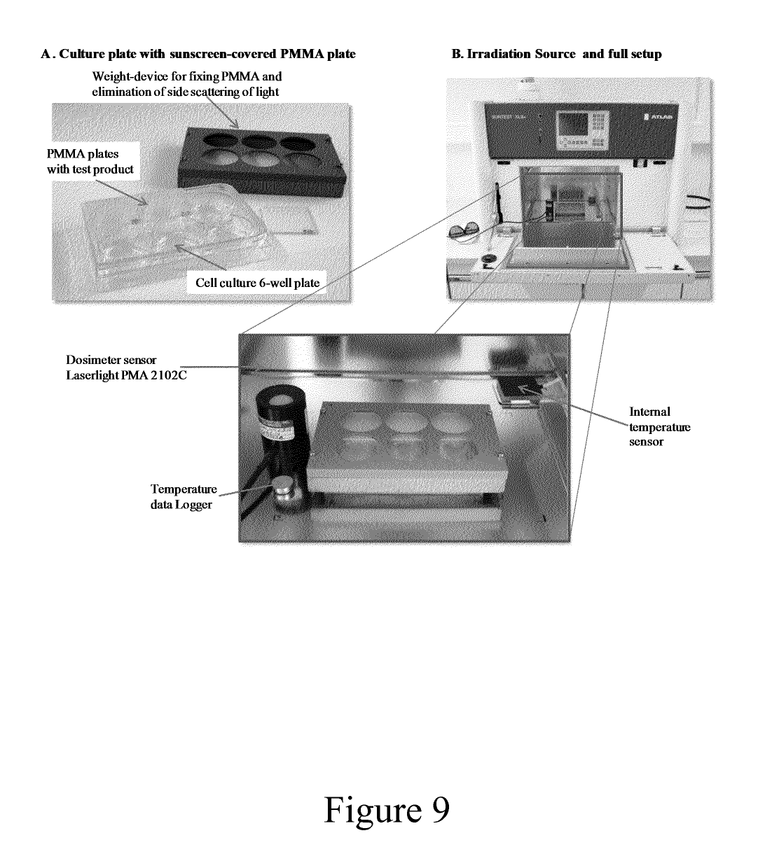

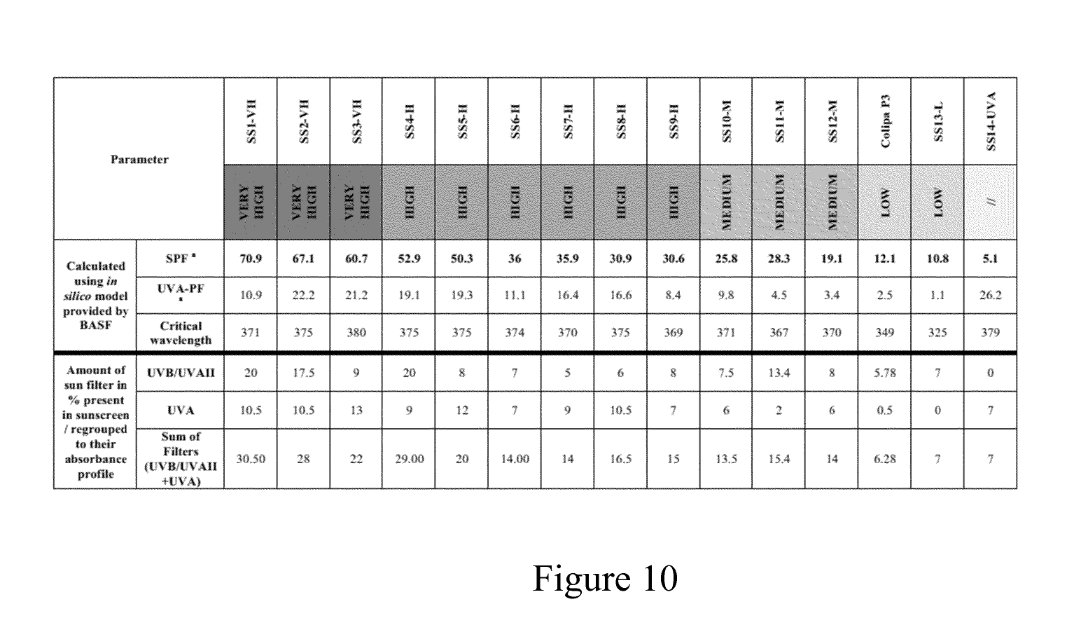

Having optimized the conditions for the cellular end-points, they were then used to evaluate the protective effects of sunscreens against UV-induced toxicity. To this end, PMMA plates were coated with sunscreens and then placed above the cells during UV irradiation (see FIG. 9). The selected cellular end-points were analysed using the optimized conditions. The sunscreens were selected so that they covered a range of UVB and UVA protection factors. The SPF protection classification of each sunscreen (cf. Table 1 appearing on FIG. 10) was done according to recommendations set out by the European Commission¹³. The percentage of the used UVAII/UVB, UVA and broad spectrum filters in the tested formulations was evaluated using the BASF Sunscreen Simulator and are schematically represented in Table 1 (cf. FIG. 10).

Effect of Sunscreens on Keratinocyte Viability

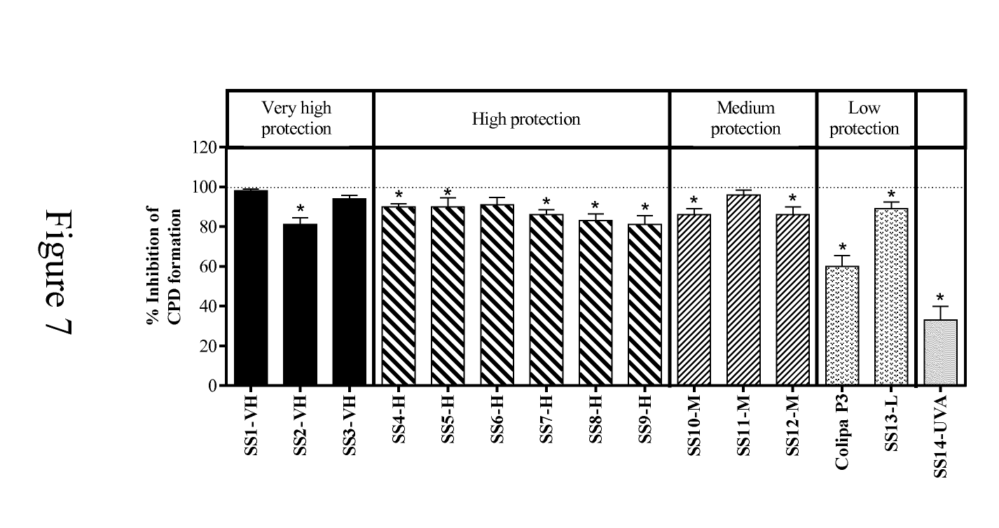

The protective effects of different sunscreens on the viability of HaCaT cells (measured using Trypan blue exclusion) were evaluated 24 h post-irradiation (FIG. 6). Doses of 200 and 828 mJ/cm² were tested; however, the differences in the protection effects were more distinct when the high UV dose was used (represented in FIG. 6). In general, sunscreens with “very high” and “high” protection were the most effective at preventing a loss in viability due to UV irradiation. Moreover, cells which had been irradiated in the presence of most of these sunscreens were almost the same viability as non-irradiated cells (i.e. ˜100%). Although high protection sunscreens prevented toxicity, the protection against UV-induced cell death did not directly correlate with the SPF of the tested sunscreen. For example, SS1-VH (SPF 70.9) and SS11-M (SPF 28.3) provided the same degree of protection against UV induced cell death (95% and 98% viability, respectively), despite the former sunscreen being classified as exhibiting “very high” protection and the latter “medium” protection. A second example is the “medium” protection sunscreen, SS10-M (SPF 25.8), which provided the same lower degree of inhibition of cell death as “high” protection classified sunscreen, SS9-H (SPF 30.6) (74% and 71% inhibition of cell death, respectively). Sunscreens classified as “low” protection (the reference sunscreen, Colipa P3 (SPF 12.1) and SS13-L (SPF 10.8) were the least effective in inhibiting cell death (FIG. 6). Colipa P3 was able to prevent the cytotoxic effects of UV irradiation only when cells were irradiated for a short time (100-200 mJ/cm², data not shown). When keratinocytes were irradiated and protected by a formulation which only filters wavelengths across the UVA spectrum (SS14-UVA, FIG. 6), the viability was the same as cultures exposed to UV irradiation without any UV protection (PPMA plates covered with glycerin).

Effect of Sunscreens on CPD Formation

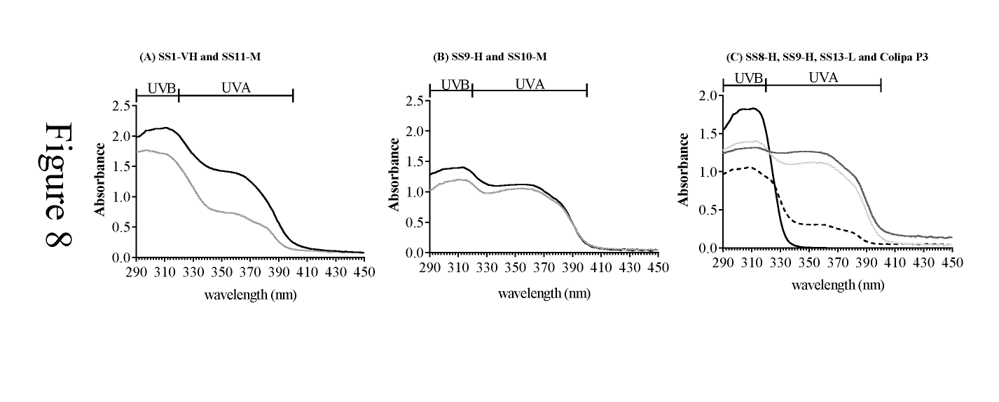

The ability of different sunscreen formulations to prevent CPD formation was evaluated 2 h post-irradiation after a dose of 828 mJ/cm². There were clear differences between the extents of inhibition by the individual UV filter formulations (FIG. 7). In keeping with the effects on viability, the best protection against CPD formation was provided by “high protection” sunscreens. Formulations that provided only filtration across the UVA spectrum inhibited CPD formation by only 33%. As with viability, the protection against UV-induced DNA damage did not directly correlate with the claimed SPF. For example, the protection by formulations classified as “high” protection with a SPF of 30 (e.g. SS8-H-SPF 30.9 and SS10-M-SPF 30.6) was marginally but significantly lower (p-values were both 0.025) than that of formulations containing only UVB filters, classified as “low” protection (SPF 10.8).

The formulation SS13-L (SPF 10.8; UVB/UVAII 7), containing the same percentage of UVB/UVAII filters as a formulation classified “high protection” (e.g. SS5-H-SPF 50.3; UVB/UVAII 8), were equally effective in inhibiting CPD formation. Similarly, the sunscreen formulation classified as “medium” protection (SS11-M-SPF 28.3; UVB/UVAII 13.4), containing a high percentage of UVB/UVAII filters and an absorption spectra comparable to that of a “high” protection sunscreen (SS5-H-SPF 50.3; UVB/UVAII 8), also inhibited CPD formation to a similar extent. Interestingly, Sunscreen “Colipa P3” (SPF 12.1; total filters percentage 6.78) and the sunscreen with only UVB filters (SS13-L, SPF 10.8; UVB/UVAII 7) are both classified as “low protection”, but they exhibited a significant difference in their ability to inhibit CPDs formation (60% and 89%, respectively).

Effect of Sunscreens on p38 Phosphorylation

The prevention of p38 MAPK activation by five sunscreens during UV irradiation was evaluated 30 min post-irradiation after a dose of 200 mJ/cm². The other sunscreens listed in table 1 (cf. FIG. 10) were not tested in this assay. Sunscreens with “very high”, “high” and “medium” protection were the most effective in preventing p38 phosphorylation (SS1-VH, SS4-H and SS10-M inhibited 90±7%, 86±14% and 84±13% of p38 phosphorylation, respectively). These sunscreens were also significantly more effective in inhibiting p38 phosphorylation compared to “low” protection sunscreens (Colipa P3, 64±12% inhibition) and the sunscreen formulation containing only UVA filters (39±3% inhibition).

Effect of Sunscreens on p53 Activation

The prevention of p53 activation by four sunscreens during UV irradiation was evaluated 6 h post-irradiation after a dose of 100 mJ/cm². The other sunscreens listed in table 1 (cf. FIG. 10) were not tested in this assay The two sunscreens with “very high” and “high” protection were the most effective in preventing p53 activation (95±12% and 96±2% inhibition of activation by SS1-VH and SS5-H, respectively). Similar to the findings from the other cellular endpoints, the “low” protection sunscreen, Colipa P3, was less effective in preventing p53 activation (87±4% inhibition) and the UVA only filter prevented only 70±16% of the UV induced p53 activation.

Applicant has developed and optimized a simple cell-based method to evaluate the photoprotection properties of a panel of sunscreens. This in vitro model combines the use of keratinocytes, basic but specific cellular endpoint plate reader assays and PMMA plates as used in the UVA-PF in vitro assay, such that UV-induced alterations to cellular pathways can be measured and the protective effects of sunscreens against these specific endpoints assessed. Applicant has avoided potential interactions between the sunscreen ingredients and the skin to rule out variability between the assays (which should be as low as possible in screening) by applying sunscreens to PMMA plates, which is also according to the in vitro COLIPA and ISO methods⁴⁻⁵. The assay is intended for higher throughput and, should a compound require further investigations as a result of this initial test, more comprehensive assays could be employed (e.g. genomics, transcriptomics, always taking the different kinetics of the evaluated endpoints into account). The basic concept of this assay has gained interest in the last year such that others have also determined the photoprotection properties of sunscreen filters using methods based on this technique—either using a single parameter to measure cytotoxicity (Neutral Red¹⁴) or using multiple measurements to compare products¹⁵ with novel ingredients. Our study extends the current knowledge and highlights a number of important aspects of the model: (1) adverse effects, which may be acute or latent, may not be detected by a single endpoint. The versatility of this model allows for the measurement of multiple endpoints to provide a more comprehensive and predictive assay; (2) different endpoints require different conditions for optimal detection e.g. radiation dose and length of incubation; (3) sequential effects of UV radiation and pathways of toxicity, as well as recovery, can be monitored by measuring different endpoints at multiple time points in the same assay; and (4) screening of a panel of sunscreens containing different amounts of UVA and UVB filters is possible, allowing for correlations between formulations and their effects to be captured in a single assay.

The measurement of endpoints can be focused towards different cellular pathways, such as cytotoxicity/apoptosis and carcinogenicity/genotoxicity, which are all adverse effects of UV radiation. Applicant measured three key targets/pathways, CPD formation, p38 phosphorylation and p53 activation, as well as apoptosis and membrane leakage in keratinocytes; whereas, others have focused on measuring cellular oxidative damage in fibroblasts caused mainly by UVA, reflecting oxidative stress, mitochondrial function and DNA damage (comet assay) and expression of two photo-ageing genes¹⁵. Therefore, this methodology is intended as a tool by which specific UV (or also other wavelengths as IR) effects can be measured and potentially attenuated by sunscreens, rather than a definitive test for the global efficacy of sunscreen products. When measuring multiple endpoints it is important to ensure that each is measured under optimal conditions to achieve the highest dynamic range and thus, sensitivity. Selecting a single time point and/or UV dose would mean some of the effects would be missed. In fact, the optimal dose and post-irradiation time point were different for each of the markers we selected, reflecting the chronological appearance of cell damage: CPD formation was best measured 2 h post-irradiation with 828 mJ/cm²; p38 phosphorylation was best measured 30 min post-irradiation with 200 mJ/cm²; and p53 activation was best measured 6 h post-irradiation with 100 mJ/cm². The time window for p53 activation was very narrow (and was only evident at the 6 h time point), by contrast, CPDs were formed within 2 h and persisted for up to 15 h. Phosphorylation of p38 occurred almost immediately after UV irradiation and persisted over the entire 2 h incubation. When measuring and interpreting changes in cellular pathways the viability of the cells should be monitored since it may change according to the time point selected. For example, doses higher than 75 mJ/cm² were much more toxic at 24 h than at 2 h post-irradiation. Lower doses may allow for repair of DNA damage and recovery from the toxic effects of the UV dose.

Once the conditions for each endpoint were optimized, the keratinocyte/PMMA in vitro model was used to evaluate the efficiency of sunscreens to prevent cytotoxicity and/or changes in cellular pathways. There was a correlation between culture viability and the formation of CPDs, such that the lower the DNA damage 2 h post irradiation in presence of a specific sunscreen, the higher is the percentage of viable cells 24 h post-irradiation. For all four endpoints measured the best protection was observed for the “very high” and “high” SPF formulations; whereas, the “low” protection UVA filter sunscreen, SS14-UVA, still had protective properties but was clearly the least effective in protecting against UV-induced effects analyzed. It is noteworthy that the observations on the effects of the sunscreens on cellular endpoints did not take into account additional directly influencing effects (e.g. composition of the formulation) and focused on the type and amount of the UV absorbers.

The protection against UV-induced cell death did not directly correlate with the calculated SPF of the tested sunscreen. For example, the “high” protection SS1-VH (SPF 70.9) and “medium” protection SS11-M (SPF 28.3) both almost completely protected the cells from UV-induced CPD formation and cell death. By contrast, a lower protection against cell death and CPD formation was afforded by the “medium” protection, SS10-M (SPF 25.8), and the “high” protection, SS9-H (SPF 30.6). In addition, the “low protection” UBV filter sunscreen, SS13-L (SPF 12.1) exhibited relatively high protection against CPD formation (inhibited by 89%); whereas, the formulation which provided only absorbance across the UVA spectrum inhibited just 33% of the CPD formation. These findings can be explained by comparing the absorption spectra of the formulations: SS1-VH and SS11-M both absorb light over the UVB wavelengths with an absorbance of >1.75 OD (FIG. 8A), indicating they absorb UVB wavelengths particularly well, independent of the labelling/classification. The filters used in SS10-M and SS9-H absorb in the spectrum to a similar level over the UVB and UVA range, with a similar curve shape and with an absorbance of <1.4 OD (FIG. 8B). This suggests that sunscreens containing UVB filters (and their applied concentrations) and absorption spectra in the range of 1.4-2 OD units provide the best protection against cell death. Sunscreens containing only UVA filters provide no protection and endpoints such as viability and CPD formation are mainly driven by UVB. Moreover, Colipa P3 provided only marginally more protection to the cells than that of the sunscreen containing only UVA filters. The relatively higher protection against UV-induced CPD formation by sunscreens containing UVB filters correlates with findings that CPD lesions are mainly due to the UVB part of the spectrum with a minor contribution of the UVA, having an essential impact on p53 mutation hot spots¹⁶⁻¹⁷ associated to the formation of specific skin cancer forms. Therefore, the presence of UVB filters in a sunscreen formulation is sufficient to guarantee certain degree of protection against this specific cellular endpoint. It should also be kept in mind that, even when the amount of the UVB/UVAII filters is comparable (e.g. SS8-H=6, SS9-H=8 and SS13-L=7), (cf. Table 1 on FIG. 10), the absorbance spectra of formulations can be different (FIG. 8C) based on the individual filters used and different formulation properties, and therefore result in different protection potencies.

As with CPD formation and cell death, p38 phosphorylation and p53 activation were inhibited by sunscreens containing UVB filters. These results indicate that sunscreens containing only UVA filters participate but cannot completely protect against DNA damage and apoptosis, causing them to be less effective than sunscreens containing only UVB filters. When these UVA and UVB filters were both combined, as required by the EU authorities, in a formulation (e.g. SS7-H, SPF 36; total filters percentage 14), the resulting percentage of inhibition of CPD formation was comparable to that of the sunscreen with only UVB filters, even if it is classified as “high” protection. Applicant has developed and optimized an expandable in vitro keratinocyte model which can be used to evaluate the protective effects of sunscreens against cellular changes caused by UV radiation. The protective effects of different ingredients of the formulations can be determined and used to develop future sunscreens. In these studies, the main protective characteristics were found to be the presence, amount and absorption spectrum of the UVB filter. This versatile cellular model can be easily adapted to include other cellular endpoint measurements, making it a promising in vitro screening tool for investigating the protective effects of sunscreen formulations against UV radiation.

Materials and Methods

Sunscreens

Ten UV filter-containing formulations and 5 marketed sunscreen products with SPF ranging from 5 to 50+ were included. Within the 15 sunscreens, a typical reference sunscreen formulation for in vivo SPF testing, Colipa P3, was included (according to the Colipa International Sun protection factor test method 2006¹⁸). The qualitative filter composition of the different sunscreens is summarized in Table 1 (cf. FIG. 10). SPF, UVA-PF, and the critical wavelengths of the products were calculated by using a Sunscreen Simulator in-silico tool¹⁹.

Cell Culture, UV Irradiation and Sunscreen Application

All data (except p53) presented here have been generated using HaCaT keratinocytes during the establishing phase of the assays; however, all products have been evaluated using NHEKs with similar outcomes. Normal Human Epidermal Keratinocytes (NHEK) (PromoCell; Heidelberg, Germany) were cultured in Keratinocyte Growth Medium 2 (Ready-to-use) from PromoCell. HaCaT cells were grown in Dulbecco's modified Eagle's medium (DMEM; Sigma) supplemented with 50 U/ml penicillin and 50 pg/ml streptomycin and 5% foetal calf serum (FCS) under an atmosphere of 95% air and 5% CO₂ at 37° C. For irradiation studies, cells were removed from culture flasks by trypsinisation and seeded into 6-wells plates (Corning, N.Y., USA). HaCaT cells were grown to ˜90-100% confluence in serum-free medium for 24 h before UV irradiation. NHEK cells were seeded at 0.5-1×10⁶ cells/well in Keratinocyte Growth Medium 2 and cultured for 6 h before replacing the medium with Keratinocyte Starving Medium (without Ca²⁺ and Supplement Mix (Promega)) and culturing overnight. Before irradiation, medium was removed from HaCaT and NHEK cultures and replaced with 4 ml phosphate-buffered saline (PBS with Ca²⁺) to avoid potential photo-sensitization effect of components in culture medium on the cells. The viability of control non-irradiated NHEKs and HaCaT cells over 24 h was unaffected by incubating them in PBS (viability >97%). In additional studies, Applicant tested whether the use of PBS affected DNA repair (CPDs) and viability and confirmed there was no difference in the two endpoints when cells were incubated in PBS and Keratinocyte Starving Medium. Any medium know in the art can be used for this test. In exemplary embodiments, Keratinocyte Starving Medium or PBS can be used. The cells were irradiated at the UV doses indicated.

Square PMMA plates, 16 cm² (from Schonberg GmbH, Hamburg, Germany), were coated on their roughened side with 9.6 μl glycerin (for control wells) or 12 mg (0.75 mg/cm², according to the 2011 Colipa UVA guideline⁴ of sunscreen and then placed on the wells of the 6-well plates during UV exposure. Immediately after irradiation, cells were incubated further at 37° C. in serum-free medium for different times. The source of UV irradiation was the CPS Atlas Plus, equipped with a 750 watt xenon arc lamp as the radiation source and a filter “B” that in the range of 290-320 nm, according to the current calibration requirements of the FDA, has an irradiation intensity of 4.02 W/m² to the sample plane. This light source provides both UVB and UVA irradiation and is similar to the spectra measured in Albuquerque (38° N) at noon on 3 Jul. 2002 and in Melbourne (38° S) at solar noon on 17 Jan. 1990¹⁰ (see FIG. 8). UV doses are indicated as mJ/cm².

Measurement of Cell Viability and Apoptosis

Cell viability was measured using Trypan blue dye exclusion using the Bio-Rad TC10™ Cell Counter (Bio-Rad) assay, according to the manufacturer's instructions. The “AnnexinV/7-AAD viability detection kit” (Beckman Coulter) was used to measure the externalization of phosphatidylserine, indicating the intermediate stages of apoptosis. Live cells do not bind Annexin V; whereas, phosphatidylserine is found on the surface of early apoptotic cells which binds the Annexin V conjugated to a fluorochrome. Late apoptotic cells start to lose membrane integrity, detected by permeability to Trypan blue dye. Briefly, after the incubation, floating cells in the supernatants and trypsinized cells were harvested, washed once in PBS and then resuspended at a concentration of 1×10⁶ cells/ml before being processed according to the manufacturer's instructions for analysis. Flow cytometry analysis was performed using a Beckman Coulter FC500 model. Cell viability and the number of apoptotic cells were calculated as a percentage of untreated controls. Results are from minimum of three independent experiments.

DNA Extraction and Cyclobutane Primidine Dimer (CPD) Measurement by Enzyme-Linked Immunosorbent Assay (ELISA)

Genomic DNA was isolated using the DNeasy kit (QIAGEN) following manufacturer's instructions and quantified by measuring absorbance at 260 nm. DNA was denatured at 100° C. for 10 min and rapidly chilled on ice and added at a concentration of 150 ng/well to polystyrene flat-bottom microtitre plate (Nunc Maxisorp) pre-coated with 0.001% protamine sulphate in PBS. After drying at 40° C., the plates were washed with PBS-Tween (0.05%) and incubated with blocking solution (4% BSA in PBS) for 10 min. The plates were incubated with the anti-thymine dimers (monoclonal anti-thymine dimer, Clone H3-Sigma) antibody (1:2000) in PBS/0.05% Tween-20, and then with an anti-mouse secondary antibody (1:2000) in PBS/0.05% Tween-20.

CPD Measurement by Fluorescence Activated Cell Sorting (FACS)

HaCaT cells were fixed with 4% formaldehyde for 10 min at room temperature (RT) and then permeabilized overnight in ice-cold 70% ethanol. Cells were then resuspended in 0.5% Triton X-100/2 M HCl for 10 min at RT. After washing with Tris-Base 1 M (pH 10) and then with PBS, HaCaT cells were incubated with 100 μl PBS-TF (4% FBS/0.25% Tween-20/PBS) containing 1 μg/ml anti-thymine dimers (Monoclonal Anti-thymine Dimer, Clone H3-Sigma) antibody. After washing twice with PBS, cells were resuspended in 100 μl PBS-TF containing Alexa-Fluor 488-coupled secondary antibody (1:100) for 1 h at RT. CPD staining was then measured using flow cytometry by quantifying the change in the x-mean fluorescence between non-irradiated and irradiated samples. For each analysis, 10,000 events were collected.

p38 Analysis

HaCaT cells were collected by centrifugation and fixed in 1% formaldehyde in PBS for 10 min at 37° C. and then 1 min on ice. The cells were then permeabilized by adding ice-cold 100% methanol to reach a final concentration of 90% (v/v). The cells were incubated for 30 min on ice and then stained with anti-phospho-p38 antibody (Beckman Coulter) in Incubation Buffer (0.5% bovine serum albumin in 1×PBS) for 60 min in the dark at RT, according to manufacturer's instruction. The cells were washed once with Incubation Buffer and then resuspended in 0.5 ml PBS for flow cytometry analysis (FC 500; Beckman Coulter). For each analysis, 15,000 events were collected.

p53 Analysis

After the incubation with NHEK cultures, floating cells in the supernatants and trypsinized cells were harvested and washed once with PBS. Proteins were isolated by adding 200 μl M-PER Mammalian Protein Extraction Reagent (Fisher Scientific AG) and 200 μl of a protease inhibitor (complete ULTRA Tablets, Mini, EDTA-free, EASYpack (Roche) to the cells. The samples were incubated at RT, with shaking at 400 rpm, for 10 min before centrifuging at 14000×g for 15 min at RT. The supernatants were removed and stored at −20° C. until analysis. Protein concentrations were measured using the Bradford assay and the samples diluted to a concentration of 50 pg/ml. The amount of p53 was analyzed using the p53 pan ELISA kit (Roche) according the manufacturer's instructions. Briefly, the samples and standards were transferred to a streptavidin-coated microtiter plate, pre-coated with anti-p53 antibody-biotin. The samples were incubated for 2 h at RT on an orbital shaker (300 rpm). The plate was washed 5 times with 300 μl washing buffer before adding 200 μl of the substrate solution into the wells. The plate was covered with foil and incubated for 10-20 min at RT on an orbital shaker (300 rpm). The stop solution (50 μl) was added and the sample was mixed. The absorbance was measured at 450 nm (reference wavelength: 690 nm) within 5 min after addition of stop solution.

Statistics

Data presented herein as mean and standard deviation (SD). Statistical significance was assessed using Student's t test, and p<0.05 was accepted as statistically significant.

### REFERENCES

1. Meunier, L. (1999) Ultraviolet light and dendritic cells. Eur. J. Dermatol. 9, 269-275.
2. Takagi, Y., H. Nakagawa, H. Kondo, Y. Takema, and G. Imokawa (2004) Decreased levels of covalently bound ceramide are associated with ultraviolet B-induced perturbation of the skin barrier. J. Invest Dermatol. 123, 1102-1109.
3. Clydesdale, G. J., G. W. Dandie, and H. K. Muller (2001) Ultraviolet light induced injury: immunological and inflammatory effects. Immunol. Cell Biol. 79, 547-568.
4. COLIPA In vitro Photoprotection Methods Task Force (2011) Method for in vitro determination of UVA protection 2011. Colipa Guidelines.
5. ISO technical committee TC217 Cosmetics (2012) Determination of sunscreen UVA photoprotection in vitro (ISO/FDIS 24443). International Organization for standardization ISO publication organs.
6. Matts, P. J., V. Alard, M. W. Brown, L. Ferrero, H. Gers-Barlag, N. Issachar, D. Moyal, and R. Wolber (2010) The COLIPA in vitro UVA method: a standard and reproducible measure of sunscreen UVA protection. Int. J. Cosmet. Sci. 32, 35-46.
7. Mitchell, D. L. and R. S. Nairn (1989) The biology of the (6-4) photoproduct. Photochem. Photobiol. 49, 805-819.
8. Chouinard, N., K. Valerie, M. Rouabhia, and J. Huot (2002) UVB-mediated activation of p38 mitogen-activated protein kinase enhances resistance of normal human keratinocytes to apoptosis by stabilizing cytoplasmic p53. Biochem. J. 365, 133-145.
9. Hildesheim, J., R. T. Awwad, and A. J. Fornace, Jr. (2004) p38 Mitogen-activated protein kinase inhibitor protects the epidermis against the acute damaging effects of ultraviolet irradiation by blocking apoptosis and inflammatory responses. J. Invest Dermatol. 122, 497-502.
10. Schwarz, M., P. A. Munzel, and A. Braeuning (2013) Non-melanoma skin cancer in mouse and man. Arch. Toxicol. 87, 783-798.
11. Diffey, B. L. (2002) Sources and measurement of ultraviolet radiation. Methods 28, 4-13.
12. Greinert, R., O. Boguhn, D. Harder, E. W. Breitbart, D. L. Mitchell, and B. Volkmer (2000) The dose dependence of cyclobutane dimer induction and repair in UVB-irradiated human keratinocytes. Photochem. Photobiol. 72, 701-708.
13. European Commission (2007) Recommendation on the efficacy of sunscreen products and the claims made relating thereto. Official Journal of the European Union (2006/647/EC) L265/40.
14. Gaspar, L. R., J. Tharmann, P. M. Maia Campos, and M. Liebsch (2013) Skin phototoxicity of cosmetic formulations containing photounstable and photostable UV-filters and vitamin A palmitate. Toxicol. In Vitro 27, 418-425.
15. Bruge, F., L. Tiano, P. Astolfi, M. Emanuelli, and E. Damiani (2014) Prevention of UVA-induced oxidative damage in human dermal fibroblasts by new UV filters, assessed using a novel in vitro experimental system. PLoS. One. 9, e83401.
16. Applegate, L. A. and E. Frenk (1995) Oxidative defense in cultured human skin fibroblasts and keratinocytes from sun-exposed and non-exposed skin. Photodermatol. Photoimmunol. Photomed. 11, 95-101.
17. Drouin, R. and J. P. Therrien (1997) UVB-induced cyclobutane pyrimidine dimer frequency correlates with skin cancer mutational hotspots in p53. Photochem. Photobiol. 66, 719-726.
18. Colipa, CTFA SA, JCIA, and CTFA (2006) International Sun Protection Factor (SPF) test method. The European Cosmetic, Toiletry and Perfumery Association.
19. Herzog B. and Osterwalder O. In Silico Determination of topical Sun Protection. Cosmetic Sience Technology, 62-70. 2011.

1. A method of evaluating a sunscreen, comprising:
(a) measuring protective effect(s) of a sunscreen and at least one cellular alteration caused by irradiation over the ultraviolet (UV), visible and infra-red (IR) wavelengths, and
(b) evaluating the sunscreen by comparing the protective effect(s) thereof against a control, wherein the comparison is based on the at least one cellular alteration.

2. The method according to claim 1, wherein the method comprises a step of exposing cells to irradiation.

3. The method according to claim 1, wherein the irradiation is a UV irradiation.

4. The method according to claim 1, wherein the method is an in vitro method.

5. The method according to claim 2, wherein cells comprise keratinocytes, fibroblasts, melanocytes or any mixture thereof.

6. The method according to claim 5, wherein cells are HaCaT cells or NHEK cells.

7. The method according to claim 1, wherein a sunscreen-coated substrate is placed between the surface of cells and the irradiation source during irradiation.

8. The method according to claim 7, wherein the substrate is a polymethylmethacrylate (PMMA) plate or a quarz plate.

9. The method according to claim 7, wherein the substrate is coated with 0.50 mg up to 1.5 mg sunscreen per cm².

10. The method according to claim 1, wherein step (a) comprises detecting, measuring or monitoring at least two cellular alterations selected from cyclobutane pyrimidine dimer (CPD) formation, p38 protein phosphorylation, p38 mitogen-activated protein kinase (MAPK) activation, p53 protein activation and cell viability or intermediate stages of apoptosis.

11. The method according to claim 10, wherein when the cellular alteration is CPD formation, the detection or measuring is performed between 2 hours and 15 hours post-irradiation at 828 mJ/cm².

12. The method according to claim 10, wherein when the cellular alteration to detect or measure is p38 protein phosphorylation or p38 mitogen-activated protein kinase (MAPK) activation, the detection or measuring is to be performed immediately after or up to two hours post-irradiation at 200 mJ/cm² irradiation.

13. The method according to claim 10, wherein when the cellular alteration is a p53 protein activation, the detection or measuring is performed 6 hours post-irradiation at 100 mJ/cm².

14. The method according to claim 10, wherein when the cellular alteration is cell viability or intermediate stage(s) of apoptosis, the detection or measuring is performed from 1 to 30 hours post-irradiation above 75 mJ/cm².

15. The method according to claim 11, wherein the detecting or measuring step is performed by ELISA.

16. The method according to claim 12, wherein the detecting or measuring step is performed by FACS.

17. The method according to claim 14, wherein the detecting, measuring or monitoring step is performed by tryptan blue exclusion or measure of the externalization of phosphatidyl serine.

18. The method according to claim 1, wherein the method is performed in parallel on several identical or different cell cultures or models, with identical or different sunscreens.

19. The method according to claim 18, wherein the method is performed in parallel under different irradiation conditions.

20. The method according to claim 18, wherein the sunscreens comprise different UVA and/or UVB filters or absorbers or different concentrations of UVA and/or UVB filters or absorbers.

21. The method according to claim 3, wherein the irradiation is UVB and/or a UVA irradiation.
